(19) United States
(12) Patent Application Publication
(10) Pub. No.: US 2004/0265835 A1
(43) Pub. Date: Dec. 30, 2004

(54) Method of sorting vesicle-entrapped, coupled nucleic acid-protein displays

(76) Inventors: David M. Lemaster, Slingerlands, NY (US); James H. Jett, Los Alamos, NM (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/610,024

(22) Filed: Jun. 30, 2003

(51) Int. Cl. C12Q 1/68; C07H 21/04; C12P 21/04; C12N 9/00; C12N 1/18

(52) U.S. Cl. 435/6; 435/69.7; 435/254.2; 435/183; 435/252.3; 435/320.1; 536/23.2

(57) ABSTRACT

A method for identifying and sorting variants of a chosen enzyme is disclosed. Enzyme variants of a chosen enzyme are obtained and then linked to their corresponding genetic code through any of a family of suitable surface display methods. The enzyme variants, now displayed on the surface of a biological particle such as a phage, virus, yeast, or bacterium are then encapsulated in a vesicle containing an enzyme activity-sensitive assay reagent. The enzyme variant is thus exposed to the assay reagent, and displays a signal using the enzyme activity-sensitive assay reagent in a manner proportionate to the levels of activity of the enzyme, thus rendering the vesicles suitable for mechanical sorting based on these levels. The vesicles are then sorted using methods known in the art to isolate those variants exhibiting possibly beneficial variations in enzyme function.

### GOVERNMENT RIGHTS

This invention was made with Government support under Contract Number W-7405-ENG-36 awarded by the United States Department of Energy to The Regents of the University of California. The Government has certain rights in the invention.

### BACKGROUND OF THE INVENTION

1. Field of the Invention

The present invention relates to a method for identifying and sorting enzyme variants in a large pool of surface expressed enzymes based on enzymatic activity. More specifically, the present invention relates to a method for identifying and sorting enzyme variants linked to their corresponding genetic code.

2. Description of Related Art

In recent years, the explosion in availability of raw genetic data has begun to shift the focus of research toward discovering and understanding gene products, their functions, and their interactions. This increased research into protein function has great importance in medical and pharmaceutical research. Beyond this, however, much work is currently being conducted in a broad spectrum of other industries to understand, manipulate, and improve enzyme function. Many see this ability as being a key to improving the manufacturing of chemical compounds in the near future. Specifically, researchers are seeking to cultivate the ability to modify the function of a known enzyme by generating a large number of variants of the enzyme and being able to efficiently assay for and isolate those enzyme variants which exhibit advanced or improved functionality for a selected desired trait.

Enzymes are seen as important biotechnology tools for performing reactions under conditions which are often far more favorable than those used in more conventional chemical syntheses. Specifically, enzymes can allow the use of more environmentally friendly (such as aqueous) reaction solutions, generate fewer hazardous byproducts, have a higher catalytic rate, and greatly reduce the amount of energy consumed in running the reaction. Many natural enzyme systems have been studied and are currently exploited in a large variety of industrial applications.

Many of the enzyme systems currently in use in industry suffer from a range of problems that stem from their natural origins and structures. Specifically, the use of naturally occurring enzymes for such chemical transformations often suffers from problems with reaction substrate specificity and with limited reaction conditions.

A first problem encountered with the use of naturally-occurring reaction systems in industrial applications is that often the chemical reaction desired to be performed by the selected naturally-occurring enzyme differs in at least one respect from the reaction in which the enzyme participates in vivo. Specifically, desired reactants may be in a different form, or may be entirely new to the enzyme. As a result of this, the reaction specificity of the enzyme must often be modified in order to carry out the desired reaction in an efficient or economically feasible manner.

A second limitation often observed when using naturally occurring enzymes in other reaction systems is that the enzymatic reactivity of natural enzymes has been evolutionarily optimized over time to function best under physiological conditions. As a result, these enzymes are often most usable when factors such as substrate concentration, pH, temperature, etc., are kept at physiological levels. This is problematic since these levels often differ substantially from commercially desirable reaction conditions. As a result, enzyme modification or accommodation of the limitations of the needed enzyme is then required.

Researchers are currently attempting to overcome these and other limitations through processes that drive the chemical “evolution” of known enzymes toward new enzyme variants exhibiting sought-after selection characteristics. In some situations, researchers seek to induce changes in the chosen enzyme that would make the enzyme more specific for the desired substrate. Additionally, variants are sought which function more efficiently in a given reaction mixture. Unfortunately, current methodologies for modifying enzymes do not allow the direct manipulation of a single molecule, and current knowledge about enzyme function does not allow the synthetic construction of entirely new, functioning enzymes from scratch. Much effort is currently being spent in creating systems in which “populations” of enzymes are subjected to selection pressures generated by researchers under conditions allowing opportunities for evolution to gradually drive the population's enzymes to greater levels of “fitness” in respect to a chosen characteristic. This process of “directed chemical evolution” is hoped to provide many new enzyme variants with properties more beneficial than those of native enzymes. Smith & Petrenko, Chem. Rev., 97, 391 410 (1997).

Directed-evolution approaches to engineering enhanced catalytic performance are characterized by labor-intensive mechanical separation of individual members of the target protein gene library and enzymatic testing. Most commonly, this entails growing individual bacterial, or other, cultures, each of which expresses a specific variant of the target protein and testing each variant protein's enzymatic activity. In order to make this more efficient, robotic high throughput assay systems have been commercially developed and, depending on cost, these systems can process 10⁴-10⁵ assays per day.

There is thus a critical need in the art to be able to efficiently engineer and assay enzymes for desired characteristics such as better reaction specificity, greater efficiency in specified reaction conditions, increased stability, reduced product inhibition, and for other characteristics required for optimal utilization under commercial reaction conditions

This area of research has received much attention, and recent advances in molecular biology have made it possible to introduce a large number of sequence variations into a gene for the purpose of generating protein variants (based on the altered gene sequence) having improved performance characteristics. Using current methods, protein libraries containing from about 10⁹ to about 10¹² variants of a target protein can be routinely and easily created. Thus, generating variants is known in the art.

A greater difficulty is posed by the problem of screening the enzyme variant libraries for a desired function or characteristic. Simply exposing enzyme variants to the desired target substrates is of little utility since it provides no way to screen out those variants which exhibit average or decreased catalytic ability.

In response to these difficulties, methods for linking phenotype and genotype in combinatorial polypeptide libraries have been developed. These methods include a set of methods collectively termed “surface display.”

Surface display techniques teach the use of a genetic fusion of the coding sequence of a target polypeptide with that of a surface protein of the particle being used. This fusion is then inserted into the chosen biological particle. Suitable particles include phage, virus, bacterium, and yeast particles. The introduction of the fusion into the genome of the particle results in the expression of both the surface protein and the target polypeptide and the subsequent display of the target polypeptide on the outer surface of the biological particle involved. Thus displayed, the target polypeptide becomes accessible for assay using a variety of methods, including methods for selecting variants of the polypeptide exhibiting a novel or selected functionality.

Surface display techniques thus render possible many more effective methods for directing the genetic development of the selected polypeptide. For this to occur, the coding sequence of the target polypeptide is first mutated by any of a number of suitable methods currently known in the art that produce a library of enzyme variants which would then be displayed on the surface of the biological particle.

One specific surface display technique is “Phage display.” Phage display is regarded by many as a very powerful technology for expressing individual variant proteins and for simultaneously testing them for performance characteristics. In this system, the genetic material of a phage (or bacterial virus) is modified so as to include a modified gene coding for a phage coat protein that is fused to the enzyme variant. The protein becomes coat-bound so as to “display” the attached enzyme variant on the outer surface of the phage. This technique makes the modified protein available for assay, while keeping it attached to the phage which contains the gene coding for its unique, modified structure. Utilization of phage display has been almost entirely based on binding assays in which an immobilized support is used to preferentially bind those protein/enzyme variants that exhibit a given property, such as the strongest binding, to a substrate. Phages which display more tightly binding variants of the target protein can thus be mechanically removed from the mixture by removing the support and then freeing the remaining variants. As a result, not only are the more tightly binding variants of the protein isolated, but more importantly, these proteins are also isolated with the phage which harbors the gene sequence for the protein/enzyme variant. This permits the easy amplification and propagation of the gene that expresses the desired product.

Other general approaches have been used to display protein variants that are directly attached to their corresponding gene sequences. A first one of these utilizes bacteria that have been modified so as to display target proteins on their cell surfaces. Another harnesses yeast cells in which proteins have been modified to display other proteins on the yeast cell surface. K. Dane Wittrup, Protein engineering by cell-surface display, Current Opinions in Biotechnology, 12:395, 397. In this approach, the C terminus of a selected protein is linked to the C-terminal anchor region of a cell wall protein such as the Flo1p protein. Schreuder et al., Immobilizing proteins on the surface of yeast cells, Trends Biotechnol., 14:115-120 (1996). This may be accomplished via glycosylphosphatidylinositol anchor linkages attached at the C terminus, or by fusion at the N or C terminus to the Aga2p-binding domain of the yeast a agglutinin mating receptor to form two disulfide bonds to the Aga1p cell-wall protein. K. Dane Wittrup, Protein engineering by cell-surface display, Current Opinions in Biotechnology, 12:395, 397; Boder & Wittrup, Yeast surface display for screening combinatorial polypeptide libraries, Nat. Biotechnol., 15:553-557 (1997). Other methods, including fusions in lactic acid bacteria, staphylococci, and tetrahymena, and even mammalian cell-surface displays have been demonstrated. Leenhouts et al., Antonie van Leeuwenhoek Int. J., 76:367-376 (1999); Gunneriusson et al., Appl. Environ. Microbiol., 65:4134-4140 (1999); Gaertig et al., Nat. Biotechnol., 17:462-465 (1999); Holmes & Al-Rubeai, J. Immunol. Methods, 230:141-147 (1999); Chesnut et al, J. Immunol. Methods, 193:17-27 (1996); and Chou et al., Biotechnol. Bioeng., 65:160-169 (1999).

Since the products of an enzymatic reaction generally diffuse away from the enzyme molecule that catalyzed that reaction, it is not readily apparent how a surface display particle-enrichment procedure based only on binding affinities can be used to test enzyme activities. Thus, there is a similarly critical need in the art to be able to efficiently detect those engineered enzyme variants which exhibit improved functionality in regard to a selected trait. Several techniques have become available in the art to attempt to fulfill this need to allow researchers to test the performance ability of modified enzymes.

One involves a mechanism-based inhibitor attached to an immobilized support. In this instance, when a target enzyme molecule reacts with this inhibitor, it becomes covalently attached to the immobilized support along with the phage on which the enzyme is displayed. The other approach involves the covalent attachment of a substrate molecule to the same particle on which the target enzyme is displayed and further attaching the substrate molecule to an immobilized support. Hence, when the tethered substrate reacts with the enzyme, the enzyme is not free to diffuse away. However, there are logistical limitations in both approaches: they are only one turnover assays (i.e., one enzyme=one reaction) and hence are ill suited for the optimization of an enzyme and its enzymatic activity.

All of these approaches are disadvantaged, however, by their reliance on mechanical separation methods to remove useful variants and/or their genetic sequence from assay solutions. Any such physical method of separation relies on contact of the variant with the binding support, and may thus miss a single phage or bacterium containing a beneficial enzyme variant. In summary, strategies exist in biotechnology to generate variants of an enzyme that are catalytically superior to the native enzyme in a particular set of conditions. However, identification and isolation of such superior enzymes is both difficult and expensive due to limitations in the current state of the art.

Accordingly, a need exists for a method that allows for the rapid and accurate identification and isolation of enzyme variants optimized catalyzing a chosen reaction.

### SUMMARY OF THE INVENTION

The method of the present invention has been developed in response to the present state of the art, and in particular, in response to the problems and needs in the art that have not yet been fully solved by currently available methods for screening enzyme variants. In accordance with the invention as shown and broadly described herein in the preferred embodiment, a method is provided for identifying enzyme variants that are catalytically optimized for a given reaction. An object of this invention is to provide a powerful novel means for generating beneficial enzyme variants through a directed selection process. These processes hold great promise for generating and isolating important new enzyme molecules for use in industry and medicine.

The present invention comprises a method for generating and isolating an enzyme variant exhibiting a desired enzyme activity including the steps of expressing an enzyme variant in a surface display particle that couples the enzyme variant to its coding nucleic acid; encapsulating the nucleic acid-coupled enzyme variant in a vesicle containing an enzyme activity-sensitive assay reagent; and sorting the vesicle based on the desired enzyme activity indicated by the enzyme activity-sensitive assay reagent. The enzyme variant may be generated using polymerase chain reaction (“PCR”) techniques or by using degenerate oligonucleotides to perform the PCR.

The enzyme variant of the invention may be coupled to a nucleic acid coding for the enzyme variant through phage display techniques. Alternatively, the enzyme variant may be coupled to a nucleic acid coding for the enzyme variant through viral expression techniques, bacterial expression techniques, and yeast expression techniques such as those mentioned above.

In some cases, the vesicle utilized in the encapsulating step comprises a liposome. Alternatively, the vesicle comprises a gel microdroplet. The enzyme activity-sensitive assay reagent may generate a fluorescent signal. This fluorescent signal may render the vesicle suitable for analysis and sorting using fluorescence-activated flow cytometry. The enzyme activity-sensitive reagent may directly generate a fluorescent signal, or instead be coupled to a reagent assay system that may then generate an initial reaction product that subsequently generates a fluorescent signal. The fluorescent signal may alternatively be generated by a second enzyme system encapsulated within the vesicle. This may render the vesicles suitable for forms of mechanical sorting.

In other embodiments of the invention, the enzyme activity-sensitive assay reagent may instead generate a proton gradient. In other related forms of the invention, pH-sensitive fluorescence indicators measure the desired enzyme activity. In others, the sorting of the vesicle for the desired enzyme activity is accomplished by measuring the fluorescence of the vesicle. This may be accomplished using fluorescence-activated flow cytometry techniques.

The present invention also includes a method for identifying variants of an enzyme having differing specific activities. This method may include the steps of obtaining a plurality of enzyme variants; coupling the enzyme variants to their corresponding genetic information in surface display particles; encapsulating said surface display particles in vesicles containing assay reagents, wherein an enzymatic reaction may occur between the enzyme variants and the assay reagents; and sorting said vesicles based on enzymatic activity.

Variants of a selected enzyme suitable for expression on the surface of an appropriate particle (e.g., phage, virus, bacteria, yeast) must be produced prior to the methods of the invention. Each new individual enzyme variant may demonstrate different enzymatic activity from the initial enzyme, especially those variants that have mutations in substrate-binding regions. Some may demonstrate activity superior to the native enzyme, while others may have poorer activity due to functionally deleterious mutations. Yet others of these variants will show no difference in enzymatic activity when compared to the native protein.

In the next step of the invention, the enzyme variants obtained prior to this are functionally coupled to their coding sequence using any one of a family of suitable techniques collectively termed “surface display.” These methods include phage display, viral display, bacterial display, and yeast display. Other such coupling methods could include cellular organelle display and a direct coupling of the protein to a ribosomally-bound mRNA molecule.

In a following step of the invention, the surface display particle containing the nucleic acid-coupled enzyme variant is encapsulated in a vesicle containing assay reagents. Optimally, each particle is encapsulated in its own individual vesicle. The vesicle serves as a barrier to create a miniature reaction vessel that prevents diffusion of the enzyme, its products, and the substrates into the suspension mixture.

In the next step, the products of the enzymatic reaction either act as a signal themselves, or are directly or indirectly coupled to the production of a signal. In many embodiments of the invention, the signals render the vesicles suitable for mechanical sorting based on the level of enzymatic activity exhibited by each individual enzyme variant trapped within each vesicle. Suitable methods of mechanical sorting may include fluorescence-activated cell sorting.

These and other objects, features, and advantages of the present invention will become more fully apparent from the following description and appended claims, or may be learned by the practice of the invention as set forth hereinafter.

### BRIEF DESCRIPTION OF THE DRAWINGS

In order that the manner in which the above recited and other advantages and objects of the invention are obtained will be readily understood, a more particular description of the invention briefly described above will be rendered by reference to the specific embodiments thereof that are illustrated in the appended drawing. Understanding that this drawing depicts only typical embodiments of the invention and is not therefore to be considered to be limiting of its scope, the invention will be described and explained with additional specificity and detail through the use of the accompanying drawing in which:

FIG. 1 is a flow diagram illustrating the method of the present invention.

### DETAILED DESCRIPTION OF THE PREFERRED EMBODIMENTS

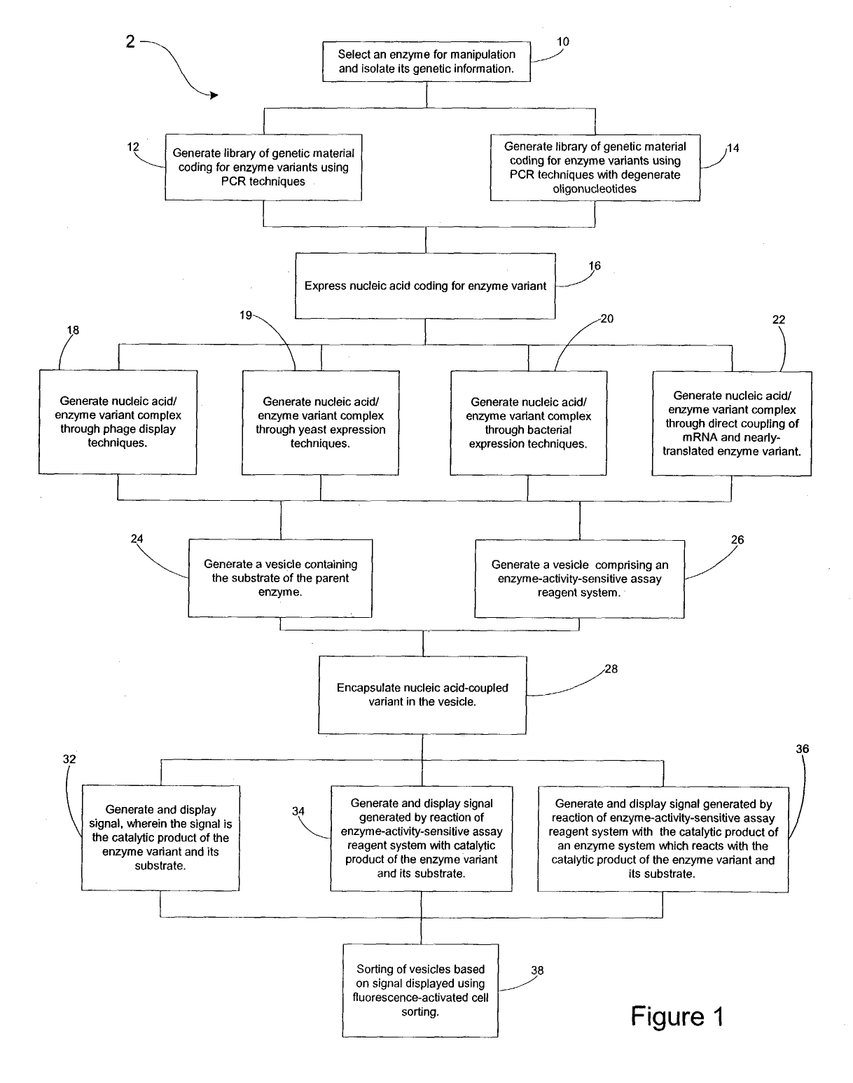

The presently preferred embodiments of the present invention will be best understood by reference to the drawing and detailed description that follow. It will be readily understood that the components of the present invention, as generally described and illustrated in the figure herein, could be arranged and designed in a wide variety of different configurations. Thus, the following more detailed description of the embodiments of the system and method of the present invention, as represented in FIG. 1, is not intended to limit the scope of the invention, as claimed, but is merely representative of presently preferred embodiments of the invention.

### Definitions

The term “surface display” is used to denote the genetic fusion of the coding sequence for a target enzyme with the coding sequence of a naturally expressed surface protein of a microbial particle such as a phage, virus, bacteria, or yeast, or on the surface of a cellular organelle. Upon expression of the naturally expressed surface protein, the enzyme becomes displayed on the surface of the particle, still attached to the surface protein. The coding sequence of the enzyme may be mutated in a variety of ways to produce a library of variants of the enzyme. These enzyme variants will be displayed on the surface of the particle, thus becoming available for assay and detection.

The term “enzyme variant” describes the protein product of a process in which the gene sequence of a selected protein is modified through PCR (with or without degenerate oligonucleotides) or other methods known in the art to generate a library of sequence variants. Many such processes are capable of generating libraries of 10⁹-10¹² copies of the gene sequence, a large percentage of which contain some sequence modification. These sequence modifications may range from simple changes such as point mutations to changes that are extremely complex in nature. Enzyme variants are produced by expressing these sequences to produce molecules which stemmed from the original enzyme, but which may contain a different sequence, and hence, possible different conformations and functionalities.

The phrase “nucleic acid coding for the enzyme variant” may encompass any sequence coding for the enzyme variant, including a DNA genetic sequence and mRNAs encoding the variant, including ribosomally-bound mRNA molecules.

The term “vesicle” denotes any closed membrane shell generated either by a physiological process such as, but not limited to, budding; or by a mechanical process such as, but not limited to, sonication. In presently preferred embodiments of the invention, liposomes are used. Liposomes are vesicles made of phospholipids that can form a lipid bilayer membrane surrounding a central aqueous compartment. Liposomes are currently used in the art to convey particles such as drugs, enzymes, vaccines, and other similar substances to targeted cells or organs. As such, they exhibit functionality that enables them to act as a microscopic reaction vessel to assay for enzymatic activity. Other non-physiological vesicles, such as those formed by many detergent-like phospholipid mimics are suitable for use in the invention.

The phrases “enzyme activity-sensitive assay reagent” or “assay reagents” are used to encompass a broad spectrum of chemical systems capable of rendering a signal proportionate to the activity level of a selected enzyme. Some such reagents may function by reacting with the catalytic product of the selected enzyme to form or activate a signal. Others will function by becoming detectable only as the substrate of the enzyme is used up by the active enzyme. These may comprise simple molecules, or they may comprise a separate enzyme system or set of enzyme systems capable of reacting to indicate the activity levels of the selected enzyme. In presently preferred embodiments of this invention, the signal given is a signal suited for the mechanical sorting of the vesicle such as a fluorescence response in a fluorescence-activated cell sorter. Fluorescence is the most presently-preferred signal since it renders the vesicles suitable for fluorescence-activated flow cytometry.

“Sorting” is used to denote a process by which vesicles displaying different levels of a signal, such as fluorescence, indicating various levels of enzyme activity, are individually analyzed and segregated into groupings, or alternatively, simply separated from other vesicles based on observed levels of enzymatic activity. In presently preferred embodiments of this invention, sorting is mechanical sorting accomplished using techniques of fluorescence-activated flow cytometry. This process is one in which cells are suspended in a manner so as to be evenly dispersed, following which they are passed in a substantially single-file manner through a laser beam by a continuous flow of a fine stream of suspension fluid. The laser light directed at the vesicle generates fluorescent light, which is emitted by the vesicle and monitored by the apparatus; and also scatters the laser light. Fluorescence-activated cell sorters are produced by companies such as Becton-Dickinson, which produces models such as the FACScan and FACStar Plus.

The phrase “phage display techniques” refers to a method of associating a selected protein with its coding sequence by fusing the coding sequence to a phage DNA coding for a phage surface protein and expressing the fusion, thus causing the selected protein to be displayed on the surface of the phage. See Phage Display, Smith, G. P., and Petrenko, V. A. Chem. Rev., 97:391 410 (1997). Specifically, phage display generally involves the insertion of the genetic code of the protein/enzyme/peptide intended for display into a phage gene coding for one of the phage coat proteins. As a result, when the viral coat protein is expressed, the chosen protein/enzyme/peptide is also expressed and is generally fused to the amino acids of the coat protein, incorporated into the coat of the phage, and subsequently displayed on the protein coat of the phage. Thus positioned, it may be exposed to the solvent around the phage, and is thus generally available to assays for functionality and activity, often behaving “essentially as it would if it were not attached to the virion surface.” Id. at 392.

Entire libraries of such phage clones may be generated, each of which may carry and express a different modified version of the targeted gene. Due to the solvent availability of the targeted protein/enzyme/peptide variant, these phage-display libraries can be screened for variants exhibiting a desired function. Techniques such as affinity purification can allow the capture of desired variants from solution, following which the variants can be produced en masse by infecting them into fresh cells and culturing the cells. This process can be varied by continually inducing additional mutations into the library population, while periodically screening for a desired function. This method, termed by some “affinity selection” serves as a type of artificial chemical selection for directing the evolution of a chemical species. Id. at 393.

The phrase “bacterial expression techniques” similarly describes a set of methods for associating a protein with its genetic coding information. In this instance, it involves inserting the gene coding for a target protein, here, a sequence for an enzyme variant, into the sequence of a bacterial membrane protein. When expressed, the product is targeted for and delivered to the bacterial cell membrane, where it may become displayed on the surface facing the solvent or the surface facing the cytosol. This manner of display renders it available for assay through compounds introduced into the solvent or the cytosol.

The term “yeast expression techniques” describes methods of tying a selected protein to its genetic information by modifying yeast cell proteins to display the selected protein on the yeast cell surface facing the solvent or cytosol. In such approaches, the C terminus of the selected protein may be linked to a cell wall protein, such as the Flo1p protein. This could be accomplished using glycosylphosphatidylinositol anchor, linkages attached at the C terminus of the protein. Alternatively, the selected protein could be fused at the N or C terminus to the Aga2p-binding domain of the yeast a agglutinin mating receptor to form two disulfide bonds to the Aga1p cell-wall protein. When expressed, the product polypeptide is targeted for and delivered to the yeast cell wall, where it becomes displayed on the surface facing the solvent. As with other surface display methods, this manner of display renders the selected protein available for assay through compounds introduced into the solvent.

The polymerase chain reaction, or “PCR” is a system for in vitro amplification of DNA. In PCR, two synthetic oligonucleotide primers, one complementary to a region on each strand of the DNA to be amplified, are added to the target DNA in the presence of an excess of nucleotides and Taq polymerase. The DNA is then repeatedly denatured (at around 90° C.), annealed to the primers (typically at 50-60° C.), and a daughter strand is extended from the primers (72° C.). In subsequent cycles, the daughter strands themselves act as templates. As a result, DNA fragments matching both primers are amplified exponentially, rather than linearly.

### Description

The instant invention describes a novel method for identifying enzyme variants having differing specific activities. This method utilizes the strengths of several biotechnology techniques to identify, isolate, and propagate enzyme variants catalytically superior to the parent enzyme that had been previously generated using techniques known in the art. One embodiment of the invention comprises an enzymatic assay system involving encapsulation of a phage display system in liposomes and the subsequent sorting of the liposomes using fluorescence analysis by flow cytometry. Other embodiments involve bacterial, viral, and yeast expression technologies, as well as mRNA binding technologies.

Referring now to FIG. 1, several of the many possible embodiments of the method of sorting vesicle-entrapped, coupled nucleic acid-protein displays (2) are displayed in the form of a flow diagram. In a first step of this invention, an enzyme is selected that exhibits a desirable property that, if modified, could be medically, pharmaceutically, or industrially more beneficial (10). The genetic material coding for this enzyme is then isolated, using methods known in the art (10). After this, a library of sequences coding for variants of the enzyme is created using methods also known and commonly used in the art such as PCR (12) or PCR using degenerate oligonucleotides (14). These nucleic acid libraries are then expressed using techniques known in the art (16).

The nucleic acid is next mechanically attached to its progeny enzyme variant through phage or viral display techniques (18), yeast expression techniques (19), bacterial expression techniques (20), or direct coupling of the mRNA and the nearly complete enzyme variant (22). Following this, a vesicle is generated which may contain either just a substrate of the parent enzyme (24), or an enzyme-activity-sensitive assay reagent (26) that may include the substrate of the parent enzyme and other reagents or systems whose activity would serve to demonstrate the catalytic activity of the enzyme variant. The various testing methods are suitable for use with sequence and variant complexes generated using any of the methods disclosed herein. The nucleic-acid coupled variant is next encapsulated in the vesicle (28). Thus encapsulated, the enzyme variant is allowed to react with the reagents/substrates encapsulated within the vesicles and generate a signal (32, 34, 36). In some embodiments, the signal is simply the intended catalytic product of the enzyme variant (32). In others, the signal is generated by the reaction of an enzyme-activity-sensitive assay reagent with the catalytic product of the enzyme variant (34). In yet others, the signal is generated by an enzyme system that reacts with the product of a first enzyme system that had reacted with the product of the enzyme variant (36). Finally, the vesicles are sorted mechanically according to the amount of signal displayed using methods known in the art such as fluorescence-activated cell sorting (38).

As briefly noted above, the method of the invention may be accordingly varied to use any of a group of suitable surface display techniques in the step of generating the nucleic acid/enzyme variant complex. Several such techniques will be discussed below.

A first such technique has been popularly termed “phage display.” Display of a number of enzymes has been reported using filamentous phage display systems that are efficiently propagated through Escherichia coli (E. Coli) bacterial hosts. Smith, Chem. Rev., 97, 391 (1997). As noted above, such bound enzymes often exhibit activity levels similar to those of the free enzyme. In addition to its genetic utility, the filamentous phage system offers significant mechanical advantages. Such assays are stable over a wide pH range from 2 (Smith, Science, 228, 1315 (1985)), to 11 (Harrison, Meth. Enzymol., 267, 93 (1996)); of temperatures (4° C.-60° C. Tan, J. Mol. Biol., 286, 787 (1999)); and are stable in the presence of organic solvents (Petrenko, Prot. Eng., 9, 797 (1996)). As a result, phage display-based enzyme assays can be carried out over a wide range of conditions without a loss of coupling to their genetic information. Furthermore, this tolerance to pH extremes will prove useful for terminating the enzymatic assays at defined time intervals, thus allowing the enzyme reaction step to potentially be carried out separately from the assay/separation step.

In these phage display systems, the nucleic acid is generally inserted into the gene coding for a coat protein of the phage in a region of the protein corresponding to the outside face of the final assembled virion such that when expressed and incorporated into a daughter phage, the desired enzyme variant is displayed on the outer surface of the phage, accessible to the solvent for assay. Viral display systems exhibit similar characteristics and abilities to the phage display systems discussed here.

Bacterial expression systems may also be used to effectively couple the protein/enzyme variant to its corresponding genetic data. In these systems, the nucleic acid coding for the variant is inserted into the gene coding for a membrane protein. Upon expression, the enzyme variant is displayed on the membrane of the bacterium and thus made available to the solvent for assay.

Similarly, yeast expression systems operating in a manner analogous to the bacterial or viral/phage display systems are also suitable for the methods of the invention. In yeast display systems, the selected protein is linked to its genetic information by modifying yeast cell surface proteins such as cell wall proteins to display the selected protein on the yeast cell surface. In some such approaches, the C terminus of the selected protein may be linked to cell wall protein Flo1p. This could be accomplished using glycosylphosphatidylinositol anchor linkages attached at the C terminus of the protein. Alternatively, the selected protein could be fused at the N or C terminus to the Aga2p-binding domain of the yeast a agglutinin mating receptor to form two disulfide bonds to the Aga1p cell-wall protein. Other similarly-expressed and targeted cell wall proteins could be suitable for the practice of the invention.

When expressed, the product polypeptide is targeted for and delivered to the yeast cell wall, where it becomes displayed on the surface facing the solvent. As with other surface display methods, this manner of display renders the selected protein available for assay through compounds introduced into the solvent.

According to the next step of the instant invention, vesicles such as liposomes are used to isolate the individual enzyme variants from the solvent. In order to circumvent exposure of potentially labile biomolecules to the organic solvents commonly used in the formation of liposomes, dehydration-rehydration protocols have been developed. Kirby, Biotechnol., 2, 979 (1984). In this procedure, liposomes are created by typical techniques. The liposomes are concentrated and then mixed with the solution that is to become entrapped in the liposomes, i.e. the assay reagents. The mixture is freeze dried and then resuspended in buffer. Rupturing and resealing of the liposomes gives rise to good efficiency in entrapment. In addition to widespread use in drug and antigen entrapment applications, entrapment of whole bacteria for vaccine applications is possible. Antimisiaris, J. Immunol. Meth., 166, 271 (1993). This technique will apply analogously to the enzyme assay application for phage display, bacterial display, or protein-RNA hybrid display systems in which a phage or bacterium displaying an enzyme variant would be entrapped within a vesicle.

Coupling of the target catalytic activity to the generation of a fluorescence signal is most straightforward when the enzyme substrate itself releases a fluorophore upon reaction. In other cases, by simultaneously entrapping a second enzyme system, it would be possible to further react the initial reaction product so as to generate a fluorogenic final product. Finally, more general approaches may prove applicable. As a wide range of enzymatic reactions give rise to release or uptake of a proton, pH sensitive fluorescence indicators can be used. Nichols, Biochem. Biophys. Acta., 596, 393 (1980). Such an approach would exploit the low permeability exhibited by liposomes so that significant equilibration of the pH across the lipid bilayer does not occur. Ceh, J. Phys. Chem., B. 102, 3030 (1998).

The liposomes thus formed can then be sorted using standard flow cytometry systems. They can withstand the mechanical stress involved when using flow cytometry. Fluorescence detection in these systems can be readily carried out on samples containing several thousand fluorophores per liposome. Fuller, Cytometry, 25, 144 (1996). Hence a single entrapped enzyme molecule with quite a poor catalytic rate of 1 s⁻¹ should give a detectable signal after an hour incubation if the reaction is coupled to an appropriate fluorophore. On the other hand, given the 5μ diameter of the liposomes described in bacterial entrapment, a linear fluorescence response can be expected for greater than 106 catalytic reactions indicating that a substantial dynamic range for the enzymatic assay system and concomitantly potential applicability to enzymes which cover a wide range of catalytic rates.

Quantitation of the amount of fluorescent product and interpretation in terms of enzymatic activity is facilitated by two considerations. The configuration of the laser detection assures that the total fluorescent product is monitored. Hence, potential complications arising from variations in the size of the liposomes and the resultant variation in the product concentration are avoided. Measuring light scatter and normalizing by particle size before making a sorting decision may alternatively overcome particle size-related complications. Additionally, the reaction rate can be determined by recording time from the beginning of the reaction along with fluorescence intensity in a flow cytometer. This reaction rate may be calculated prior to making sort decisions and will be helpful in long sorts.

Using the most common approach to protein display in the filamentous phage system, a maximum of five copies of the target enzyme can be displayed on the surface of the phage. Other known approaches can be used to reduce this to a maximum of one enzyme per phage. Independent monitoring of the number of phage particles per liposome can be readily incorporated so as to eliminate liposomes containing more than one phage particle. As a result, moderately accurate specific activity measurements are feasible.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

We claim:

1. A method for isolating an enzyme variant exhibiting a selected enzyme activity comprising the steps of:
a) coupling an enzyme variant to a nucleic acid coding for the enzyme variant, thus forming a nucleic acid-coupled enzyme variant;
b) encapsulating the nucleic acid-coupled enzyme variant in a vesicle containing an enzyme activity-sensitive assay reagent; and
c) sorting the vesicle based on the desired enzyme activity indicated by the enzyme activity-sensitive assay reagent.

2. The method of claim 1, wherein the enzyme variant is coupled to a nucleic acid coding for the enzyme variant through surface display.

3. The method of claim 1, wherein the enzyme variant is coupled to a nucleic acid coding for the enzyme variant through phage display.

4. The method of claim 1, wherein the enzyme variant is coupled to a nucleic acid coding for the enzyme variant through bacterial expression.

5. The method of claim 1, wherein the enzyme variant is coupled to a nucleic acid coding for the enzyme variant through viral display.

6. The method of claim 1, wherein the enzyme variant is coupled to a nucleic acid coding for the enzyme variant through yeast display.

7. The method of claim 1, wherein the enzyme variant is coupled to a nucleic acid coding for the enzyme variant, said nucleic acid coding for the enzyme variant being a ribosomally-bound mRNA molecule.

8. The method of claim 1, wherein the vesicle comprises a liposome.

9. The method of claim 1, wherein the vesicle comprises a phospholipid mimic.

10. The method of claim 1, wherein the enzyme activity-sensitive assay reagent is a fluorescent signal.

11. The method of claim 10, wherein the fluorescent signal renders the vesicle suitable for sorting using fluorescence-activated flow cytometry.

12. The method of claim 1, wherein the enzyme activity-sensitive assay reagent comprises an enzyme system that may interact with the enzyme variant to generate a fluorescent signal.

13. The method of claim 12, wherein the fluorescent signal renders the vesicle suitable for sorting using fluorescence-activated flow cytometry.

14. The method of claim 1, wherein the enzyme activity-sensitive reagent generates an initial reaction product that subsequently generates a fluorescent signal.

15. The method of claim 14, wherein the fluorescent signal is generated by a second enzyme system encapsulated within the vesicle.

16. The method of claim 15, wherein the selected enzyme activity is measured by pH-sensitive fluorescence indicators.

17. The method of claim 1 wherein the enzyme activity-sensitive assay reagent generates a proton gradient.

18. The method of claim 1, wherein the sorting of the vesicle for the selected enzyme activity is accomplished by measuring the fluorescence of the vesicle.

19. The method of claim 1, wherein the sorting of said vesicle comprises the use of fluorescence-activated flow cytometry.

20. A method for sorting variants of an enzyme having differing specific activities comprising:
a) coupling a set of enzyme variants to their corresponding genetic information to form gene-coupled enzyme variants;
b) encapsulating said gene-coupled enzyme variants in vesicles containing assay reagents, wherein an enzymatic reaction occurs between the enzyme variants and the assay reagents thus generating a signal; and
c) sorting said vesicles based on enzymatic activity.

21. The method of claim 20, wherein the enzyme variants are coupled to their corresponding genetic information through surface display.

22. The method of claim 20, wherein the enzyme variants are coupled to their corresponding genetic information through phage display.

23. The method of claim 20, wherein the enzyme variants are coupled to their corresponding genetic information through bacterial expression.

24. The method of claim 20, wherein the enzyme variants are coupled to their corresponding genetic information through viral display.

25. The method of claim 20, wherein the enzyme variants are coupled to their corresponding genetic information through yeast display.

26. The method of claim 20, wherein the enzyme variant is coupled to a nucleic acid coding for the enzyme variant, said nucleic acid coding for the enzyme variant being a ribosomally-bound mRNA molecule.

27. The method of claim 20, wherein the vesicle comprises a liposome.

28. The method of claim 20, wherein the vesicle comprises a phospholipid mimic.

29. The method of claim 20, wherein the enzymatic reaction generates a fluorescent signal.

30. The method of claim 29, wherein the sorting of the vesicles is accomplished using fluorescence activated cell-sorting.

31. The method of claim 20, wherein the signal generated by the enzymatic reaction is the substrate for a second catalyzed reaction that generates a fluorescent signal.

32. The method of claim 31, wherein the sorting of the vesicles is accomplished using fluorescence activated cell-sorting.

33. A method for generating and isolating enzyme variants exhibiting a selected activity comprising the steps of:
a) generating a plurality of enzyme variants through PCR;
b) coupling the enzyme variants to their corresponding genetic information through phage display;
c) encapsulating said gene-coupled enzyme variants in vesicles containing assay reagents, wherein an enzymatic reaction occurs between the enzyme variants and the assay reagents, thus generating a fluorescent signal; and
d) sorting said vesicles using fluorescence activated cell cytometry.

34. A method for generating and isolating enzyme variants exhibiting a selected activity comprising the steps of:
a) generating a plurality of enzyme variants;
b) coupling the enzyme variants to their corresponding genetic information through bacterial expression;
c) encapsulating said gene-coupled enzyme variants in vesicles containing assay reagents, wherein an enzymatic reaction occurs between the enzyme variants and the assay reagents, thus generating a fluorescent signal; and
d) sorting said vesicles using fluorescence activated cell cytometry.
